(19) United States
(12) Patent Application Publication
(10) Pub. No.: US 2013/0203778 A1
(43) Pub. Date: Aug. 8, 2013

(54) PHARMACEUTICAL COMPOSITION COMPRISING A PYRIMIDINEONE DERIVATIVE

(71) Applicants: Ulhas Dhuppad, Maharashtra (IN); Sunil Chaudhari, Maharashtra (IN)

(72) Inventors: Ulhas Dhuppad, Maharashtra (IN); Sunil Chaudhari, Maharashtra (IN)

(73) Assignee: Glenmark Pharmaceuticals S.A., La Chaux-de-fonds (CH)

(21) Appl. No.: 13/579,739

(22) PCT Filed: Mar. 21, 2011
(86) PCT No.: PCT/IB11/00605

§ 371 Date: Oct. 24, 2012

Related U.S. Application Data
(60) Provisional application No. 61/317,090, filed on Mar. 24, 2010.

(30) Foreign Application Priority Data
Mar. 22, 2010 (IN) 757/MUM/2010

(51) Int. Cl. C07D 513/04

(52) U.S. Cl. CPC C07D 513/04
USPC 514/259.5

(57) ABSTRACT

The present patent application relates to a pharmaceutical composition comprising a fused pyrimidineone derivative having transient receptor potential modulating activity and a hydrophilic carrier.

### PRIORITY

This patent application claims priority to Indian provisional patent application number 757/MUM/2010 (filed on Mar. 22, 2010) and U.S. provisional patent application No. 61/317,090 (filed on Mar. 24, 2010), the contents of each of which are incorporated by reference herein.

### TECHNICAL FIELD

The present patent application relates to a pharmaceutical composition comprising a pyrimidineone derivative. Particularly, the present patent application relates to a pharmaceutical composition comprising a fused pyrimidineone derivative having transient receptor potential modulating activity and a hydrophilic carrier.

### BACKGROUND

Transient receptor potential (TRP) channels are cation channels that are permeable to monovalent and divalent cations. TRP channels are one large family of non-selective cation channels that function to help regulate ion flux and membrane potential. The TRP family consists of 6 sub-families including the transient receptor potential vanilloid-type (TRPV) channels. Transient receptor potential vanniloid-type-3 receptor (TRPV3 receptor) is one such member of the TRPV sub-family. Modulators of TRPV3 receptors and compositions of such modulators have been described in the US Patent Application Publication Nos. US 2006/0270688 and US 2007/0213321.

### SUMMARY

Co-assigned PCT Application Publication No. WO2009130560 (“the '560 application”), which is incorporated herein by reference in its entirety and for the purpose of disclosing the compounds at issue and methods of their preparation, discloses certain pyrimidineone derivatives, which are believed to modulate TRPV3 receptors, and may be useful for the treatment of various types of disease conditions implicating TRPV3 receptors including inflammation and pain.

The present invention relates to a pharmaceutical composition comprising a poorly water-soluble pyrimidineone derivative and a hydrophilic carrier.

The '560 application discloses a novel class of pyrimidineone derivatives of formula (I),

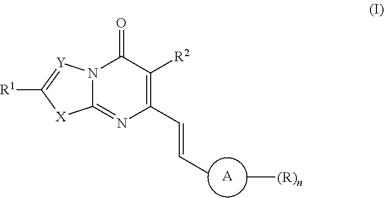

wherein X is S or NR^{b};

Y is CR³;

ring A is aryl, or heteroaryl;

at each occurrence, R, which may be the same or different, is selected from hydrogen, nitro, cyano, halogen, —OR^{a}, alkyl, alkenyl, haloalkyl, cyanoalkyl, or cyanoalkyloxy;

R¹ and R³, which may be the same or different, are each independently selected from hydrogen, halogen, nitro, cyano, —COOH, alkyl, alkenyl, alkynyl, or haloalkyl; or R¹ and R³ together with the carbon atoms to which they were attached may form a 5 to 7 membered cyclic ring, which may be substituted or unsubstituted, saturated, unsaturated or partially saturated, which cyclic ring may optionally contain one or more heteroatoms selected from O, NR^{b} or S;

R² is aryl, or heteroaryl, each of which may be optionally mono- or polysubstituted with substituent(s) independently selected from the group consisting of halogen, hydroxyl, nitro, cyano, —COOH, —NR⁴R⁵, acyl, alkyl, alkenyl, alkoxy, cyanoalkoxy, haloalkyl, haloalkyloxy, cycloalkyl, cycloalkylalkyl and, cycloalkylalkoxy;

at each occurrence, R^{a}, which may be the same or different, is selected from the group consisting of hydrogen, alkyl, haloalkyl, cyanoalkyl, alkenyl, cycloalkyl, alkoxyalkyl, cycloalkylalkyl, substituted or unsubstituted arylalkyl, heteroarylalkyl, and heterocyclylalkyl;

at each occurrence, R^{b} is selected from hydrogen, alkyl, or arylalkyl;

at each occurrence, R⁴ and R⁵, which may be the same or different, are independently selected from hydrogen, alkyl, alkenyl, cycloalkyl, cycloalkylalkyl, cycloalkenyl, arylalkyl, heteroarylalkyl, or heterocyclylalkyl; and

‘n’ is an integer ranging from 0 to 5, inclusive;

or a pharmaceutically acceptable salt thereof.

The '560 application discloses inter alfa, certain pyrimidineone derivatives viz., 7-{(E)-2-[2-(cyclopropylmethoxy)-3-methoxyphenyl]vinyl}-6-(4-trifluoromethoxyphenyl)-5H-[1,3]thiazolo-[3,2-a]pyrimidin-5-one (synonymously, “Compound I”) or its salt; 7-{(E)-2-[2-(cyclopropylmethoxy)-3-methoxyphenyl]vinyl}-6-(4-trifluoromethylphenyl)-5H-[1,3]thiazolo[3,2-a]pyrimidin-5-one (synonymously “Compound II”) or its salt; 4-{7-[(E)-2-(3-methoxy-2-neopentyoxy)phenyl)-1-ethenyl]-5-oxo-5H-[1,3]thiazolo[3,2-a]pyrimidin-6-yl}benzonitrile (synonymously, “Compound III”) or its salt; 7-[(E)-2-{3-methoxy-2-neopentyloxyphenyl)-1-ethenyl-6-[4-trifluoromethoxy)phenyl]-5H-[1,3]thiazolo[3,2-a]pyrimidin-5-one (synonymously, “Compound IV”) or its salt; and 7-[(E)-2-{3-methoxy-2-neopentyloxyphenyl)-1-ethenyl-6-[4-trifluoromethylphenyl]-5H-[1,3]thiazolo[3,2-a]pyrimidin-5-one (synonymously, “Compound V”) or its salt.

Some of the pyrimidineone derivatives of formula (I) have been found to be poorly soluble in water. For example, the inventors of the present invention have particularly found that Compound I was found to be practically insoluble in water (aqueous media having different pH ranging over 1.2 to 6.8). Hence, the inventors of the present invention believe that pharmaceutical compositions comprising a pyrimidineone derivative of formula (I) including Compound I, Compound II, Compound III, Compound IV and Compound V or salts thereof, and a hydrophilic carrier would help to improve the solubility, in vitro dissolution and hence bioavailability of these compounds in a subject. Such pharmaceutical compositions that include a solid dispersion comprising a poorly water-soluble pyrimidineone derivative and a hydrophilic carrier are contemplated herein.

Thus, in an embodiment, the present invention provides a pharmaceutical composition comprising an active ingredient selected from a group consisting of 7-{(E)-2-[2-(cyclopropylmethoxy)-3-methoxyphenyl]vinyl}-6-(4-trifluoromethoxy phenyl)-5H-[1,3]thiazolo-[3,2-a]pyrimidin-5-one (“Compound I”) or 7-{(E)-2-[2-(cyclopropylmethoxy)-3-methoxyphenyl]vinyl}-6-(4-trifluoromethylphenyl)-5H-[1,3]thiazolo[3,2-a]pyrimidin-5-one (“Compound II”) or 4-{7-[(E)-2-(3-methoxy-2-neopentyoxy)phenyl)-1-ethenyl]-5-oxo-5H-[1,3]thiazolo[3,2-a]pyrimidin-6-yl}benzonitrile (“Compound III”) or 7-[(E)-2-{3-methoxy-2-neopentyloxyphenyl)-1-ethenyl-6-[4-trifluoromethoxy)phenyl]-5H-[1,3]thiazolo[3,2-a]pyrimidin-5-one (“Compound IV”) or 7-[(E)-2-{3-methoxy-2-neopentyloxyphenyl)-1-ethenyl-6-[4-trifluoromethylphenyl]-5H-[1,3]thiazolo[3,2-a]pyrimidin-5-one (“Compound V”) or salts thereof; and a hydrophilic carrier. Preferably, the active ingredient is 7-{(E)-2-[2-(cyclopropylmethoxy)-3-methoxyphenyl]vinyl}-6-(4-trifluoromethoxy phenyl)-5H-[1,3]thiazolo-[3,2-a]pyrimidin-5-one (“Compound I”) or its salt.

In another embodiment, the present invention provides a pharmaceutical composition comprising a solid dispersion that includes Compound I or its salt; and a hydrophilic carrier.

The hydrophilic carrier, in the context of present invention, includes a surfactant, complexing agent, cosolvent, polymer and mixtures thereof. Preferably, the hydrophilic carrier includes a surfactant, a polymer or mixtures thereof.

In an embodiment, the present invention relates to the pharmaceutical composition, wherein the weight ratio of the active ingredient to a hydrophilic carrier from about 1:0.1 to about 1:100.

In another embodiment, the present invention relates to the pharmaceutical composition, wherein the weight ratio of the active ingredient to a hydrophilic carrier ranges from about 1:0.5 to about 1:50. Preferably, in the pharmaceutical composition, the weight ratio of the active ingredient to the hydrophilic carrier ranges from about 1:1 to about 1:20.

In an embodiment, the present invention provides a pharmaceutical composition comprising a solid dispersion that includes Compound I or its salt; and a hydrophilic carrier, wherein the weight ratio of the Compound I or its salt to the hydrophilic carrier ranges from about 1:1 to about 1:10.

In another embodiment, the present invention relates to a pharmaceutical composition comprising a solid dispersion that includes Compound I or its salt, poloxamer, hydroxypropylmethyl cellulose and lauroyl macrogolglycerides.

In a further embodiment, the present invention relates to a pharmaceutical composition comprising a solid dispersion that includes Compound I or its salt, poloxamer, hydroxypropylmethyl cellulose and lauroyl macrogolglycerides in the weight ratio of about 1:1:1:2, respectively.

In yet another embodiment, the present invention, relates to a pharmaceutical composition wherein, the active ingredient is present in an amount ranging from about 1% w/w to about 70% w/w. Preferably, the active ingredient is present in an amount ranging from about 1% w/w to about 50% w/w and more preferably ranging from about 5% w/w to about 25 w/w.

In a specific embodiment, the present invention relates to a pharmaceutical composition comprising from about 5% w/w to about 25 w/w of Compound I or its salt as active ingredient, from about 5% w/w to about 25% w/w poloxamer, from about 5% w/w to about 25% w/w hydroxypropylmethyl cellulose; and from about 5% w/w to about 50% w/w lauroyl macrogolglycerides.

In an embodiment, the present invention relates to a pharmaceutical composition for oral administration comprising Compound I or its salt, and a hydrophilic carrier; wherein the pharmaceutical composition releases at least 75% of the contained Compound I or its salt within 60 minutes when tested in USP apparatus type II containing 900 mL of 0.1N hydrochloric acid (HCl) with 1% (w/v) SLS maintained at a temperature of about 37±0.5° C., and stirred at 75 rpm. Preferably, the composition releases 85% of the contained Compound I or its salt under the stipulated conditions.

In another embodiment, the present invention relates to a pharmaceutical composition, wherein the Compound I or its salt is present in partially amorphous form. In an embodiment, the pharmaceutical composition contains at least about 10% of the contained Compound I or its salt in amorphous form. Preferably, the pharmaceutical composition contains from about 10% to about 50%, or more preferably from about 15% to about 40%, of the contained Compound I or its salt in amorphous form.

In an embodiment, the present invention relates to a method of treating a disease condition associated with TRPV3 receptor modulation in a subject, wherein the method includes administering the subject a pharmaceutical composition that includes an active ingredient selected from Compound I, Compound II, Compound III, Compound IV and Compound V or salt thereof, and a hydrophilic carrier. Preferably, the active ingredient is Compound I or its salt.

In another embodiment, the present invention relates to a method of treating a disease condition associated with TRPV3 receptor modulation in a subject, wherein the method includes administering the subject a pharmaceutical composition comprising a solid dispersion that includes Compound I or its salt; and a hydrophilic carrier.

In another embodiment, the present invention contemplates use of a pharmaceutical composition for the treatment of disease condition associated with TRPV3 receptor modulation in a subject comprising administering to the subject a pharmaceutical composition that includes a solid dispersion comprising Compound I or its salt and a hydrophilic carrier.

In a further embodiment, the present invention relates to a pharmaceutical composition for the treatment of disease condition associated with TRPV3 receptor modulation in a subject comprising administering to the subject a composition comprising a solid dispersion that includes Compound I or its salt and a hydrophilic carrier.

In yet another embodiment, the present invention provides a process for the preparation of a pharmaceutical composition, said process comprising preparing a solid dispersion of the Compound I or its salt and a hydrophilic carrier; and formulating the solid dispersion in a suitable dosage form. The process comprises preparing a solid dispersion of the Compound I or its salt and a hydrophilic carrier, converting this solid dispersion in a granule formulation and formulating the granules into a suitable dosage form for oral administration.

### BRIEF DESCRIPTION OF THE DRAWING

FIG. 1 represents X-ray diffraction data of Compound I.

FIG. 2 represents X-ray Diffraction pattern of placebo granule composition of Example 7.

FIG. 3 represents the X-ray diffraction data of the granule composition of Example 7.

FIG. 4 represents the X-ray diffraction data of the pharmaceutical composition of Example 11.

### DETAILED DESCRIPTION

The present invention relates to a pharmaceutical composition comprising a solid dispersion that includes a poorly water-soluble pyrimidineone derivative of formula (I) and a hydrophilic carrier.

The terms used herein are defined as follows. If a definition set forth in the present application and a definition set forth earlier in a provisional application from which the priority is claimed are in conflict, the definition in the present application shall control the meaning of the terms.

The '560 application discloses inter alia, certain pyrimidineone derivatives viz., 7-{(E)-2-[2-(cyclopropylmethoxy)-3-methoxyphenyl]vinyl}-6-(4-trifluoromethoxyphenyl)-5H-[1,3]thiazolo-[3,2-a]pyrimidin-5-one (synonymously, “Compound I”) or its salt; 7-{(E)-2-[2-(cyclopropylmethoxy)-3-methoxyphenyl]vinyl}-6-(4-trifluoromethylphenyl)-5H-[1,3]thiazolo[3,2-a]pyrimidin-5-one (synonymously “Compound II”) or its salt; 4-{7-[(E)-2-(3-methoxy-2-neopentyoxy)phenyl)-1-ethenyl]-5-oxo-5H-[1,3]thiazolo[3,2-a]pyrimidin-6-yl}benzonitrile (synonymously, “Compound III”) or its salt; 7-[(E)-2-{3-methoxy-2-neopentyloxyphenyl)-1-ethenyl-6-[4-trifluoromethoxy)phenyl]-5H-[1,3]thiazolo[3,2-a]pyrimidin-5-one (synonymously, “Compound IV”) or its salt; and 7-[(E)-2-{3-methoxy-2-neopentyloxyphenyl)-1-ethenyl-6-[4-trifluoromethylphenyl]-5H-[1,3]thiazolo[3,2-a]pyrimidin-5-one (synonymously, “Compound V”) or its salt.

Particularly, Compound I has been found to be practically insoluble in water (i.e., aqueous media having different pH ranging over 1.2 to 6.8), slightly soluble in methanol, very slightly soluble in ethanol, and freely soluble in acetone, methylene dichloride and chloroform.

In the present context, the term “solid dispersion” denotes a formulation wherein an active ingredient is dispersed in a molecular state or in the form of fine particles in a hydrophilic carrier domain. The solid dispersion in the context of present invention improves the solubility (and, in turn, the dissolution rate) of the active ingredient.

The pyrimidineone derivative of the present invention comprising Compound I, Compound II, Compound III, Compound IV and Compound V can be in amorphous form or crystalline form or mixtures thereof.

The term “active ingredient” (used interchangeably with “active” or “active substance” or “drug”) used herein includes pyrimidineone derivatives selected from the group comprising Compound I, Compound II, Compound III, Compound IV and Compound V, including their one or more salts, analogs, derivatives, polymorphs, solvates, single isomers, enantiomers, metabolites, prodrugs and mixtures thereof.

The term “treating” or “treatment” as used herein also covers the “prophylaxis”, “mitigation”, “prevention”, “amelioration”, or “suppression” of a disease condition in a subject.

The term “TRPV3 receptor modulation” as used herein also covers inhibition, antagonism, inverse agonism, agonism, inverse antagonism and activation of the TRPV3 receptor.

The term “subject” includes mammals like human and other animals, such as domestic animals (e.g., household pets including cats and dogs) and non-domestic animals (such as wildlife). Preferably, the subject is a human.

In an embodiment, the present invention provides a pharmaceutical composition comprising an active ingredient selected from a group consisting of 7-{(E)-2-[2-(cyclopropylmethoxy)-3-methoxyphenyl]vinyl}-6-(4-trifluoromethoxyphenyl)-5H-[1,3]thiazolo-[3,2-a]pyrimidin-5-one (Compound I) or 7-{(E)-2-[2-(cyclopropylmethoxy)-3-methoxyphenyl]vinyl}-6-(4-trifluoromethylphenyl)-5H-[1,3]thiazolo[3,2-a]pyrimidin-5-one (Compound II) or 4-{7-[(E)-2-(3-methoxy-2-neopentyoxy)phenyl)-1-ethenyl]-5-oxo-5H-[1,3]thiazolo[3,2-a]pyrimidin-6-yl}benzonitrile (Compound III) or 7-[(E)-2-{3-methoxy-2-neopentyloxyphenyl)-1-ethenyl-6-[4-trifluoromethoxy) phenyl]-5H-[1,3]thiazolo[3,2-a]pyrimidin-5-one (Compound IV) or 7-[(E)-2-{3-methoxy-2-neopentyloxyphenyl)-1-ethenyl-6-(4-trifluoromethylphenyl]-5H-[1,3]thiazolo[3,2-a]pyrimidin-5-one (Compound V) or salt thereof; and a hydrophilic carrier. Preferably the active ingredient is 7-{(E)-2-[2-(cyclopropylmethoxy)-3-methoxyphenyl]vinyl}-6-(4-trifluoromethoxyphenyl)-5H-[1,3]thiazolo-[3,2-a]pyrimidin-5-one (Compound I) or its salt.

In another embodiment, the present invention provides a pharmaceutical composition comprising a solid dispersion that includes Compound I or its salt and; and a hydrophilic carrier.

The pharmaceutical compositions of the invention include those for oral, parenteral, transdermal, transmucosal and nasal administration, among others.

The pharmaceutical compositions for oral administration may be in various forms, for example, tablets, capsules, granules (synonymously, “beads” or “particles” or “pellets”), solution, suspensions, emulsions, powders, dry syrups, and the like. The capsules may contain granule/pellet/particle/mini-tablets/mini-capsules containing the active ingredients.

The pharmaceutical compositions for parenteral administration include but are not limited to solutions for intravenous, subcutaneous or intramuscular injection/infusion, suspensions for intramuscular or subcutaneous injection, emulsions for intramuscular or subcutaneous injection and implants.

The pharmaceutical compositions for transdermal or transmucosal administration include but are not limited to patches, gels, creams, ointments and the like.

In an embodiment, the present invention relates to the pharmaceutical composition, wherein the weight ratio of active ingredient to a hydrophilic carrier ranges from about 1:0.1 to about 1:100.

In another embodiment, the present invention relates to the pharmaceutical composition, wherein the weight ratio of the active ingredient to a hydrophilic carrier ranges from about 1:0.5 to about 1:50. Preferably, in the pharmaceutical composition, the weight ratio of the active ingredient to the hydrophilic carrier ranges from about 1:1 to about 1:20.

In an embodiment, the present invention encompasses a pharmaceutical composition comprising a solid dispersion that includes an active ingredient Compound I or its salt; and a hydrophilic carrier, wherein the weight ratio of the Compound I or its salt to the hydrophilic carrier ranges from about 1:1 to about 1:10.

In another embodiment, the present invention relates to a pharmaceutical composition comprising a solid dispersion that includes Compound I or its salt, poloxamer, hydroxypropylmethyl cellulose and lauroyl macrogolglycerides.

In a further embodiment, the present invention relates to a pharmaceutical composition comprising a solid dispersion that includes Compound I or its salt, poloxamer, hydroxypropylmethyl cellulose and lauroyl macrogolglycerides in the weight ratio of about 1:1:1:2, respectively.

In yet another embodiment, the present invention relates to a pharmaceutical composition wherein, the active ingredient is present in an amount ranging from about 1% w/w to about 70% w/w. Preferably, the active ingredient is present in an amount ranging from about 1% w/w to about 50% w/w and more preferably ranging from about 5% w/w to about 25% w/w.

In a specific embodiment, the present invention relates to a pharmaceutical composition comprising from about 5% w/w to about 25% w/w of Compound I or its salt as active ingredient, from about 5% w/w to about 25% w/w poloxamer, from about 5% w/w to about 25 w/w hydroxypropylmethyl cellulose; and from about 5% w/w to about 50 w/w lauroyl macrogolglycerides.

The term “hydrophilic carrier”, refers to one or more of those pharmaceutical excipients which when admixed with a compound selected from the group comprising Compound I, Compound II, Compound III, Compound IV and Compound V, increase the aqueous solubility of the compound; and typically includes surfactant, complexing agent, cosolvent, polymer and mixtures thereof.

The hydrophilic carrier, in the context of present invention, includes surfactant, complexing agent, cosolvent, polymer and mixtures thereof. Preferably, the hydrophilic carrier includes a surfactant, a polymer or mixtures thereof.

The surfactants suitable for use in this invention include but are not limited to, poloxamer, cetrimide, cetyl trimethyl ammonium bromide (CTAB), polyoxyethylene sorbitan esters (known as POLYSORBATE or TWEEN), polyethoxylated castor oil (CREMOPHOR), methyl glucose sesquistearate, PEG-20 methyl glucoside sesquistearate, Steareth-21, polyethylene glycol 20 sorbitan monostearate, polyethylene glycol 60 sorbitan monostearate, polyethylene glycol 80 sorbitan monostearate, Steareth-20, Ceteth-20, PEG-100 stearate, sodium stearoyl sarcosinate, hydrogenated lecithin, sodium cocoylglyceryl sulfate, sodium stearyl sulfate, sodium stearoyl lactylate, PEG-20 glyceryl monostearate, sucrose monostearate, sucrose polystearates, polyglyceryl 10 stearate, polyglcyeryl 10 myristate, steareth 10, DEA oleth 3 phosphate, DEA oleth 10 phosphate, PPG-5 Ceteth 10 phosphate sodium salt, PPG-5 Ceteth 10 phosphate potassium salt, steareth-2, PEG-5 soya sterol oil, PEG-10 soya sterol oil, diethanolamine cetyl phosphate, polyglyceryl-6-dioleate (Pleurol Olique CC 497), polyethylene glycol 15 hydroxy stearate (Solutol HS 15), diethylene glycol monoethyl ether (Transcutol P), propylene glycol dicaprylocaprate (Labrafac PG), dioctyl ester of sodium sulfosuccinate bis-(2-ethylhexyl) sulfosuccinate (Docusate Sodium), propylene glycol monocaprylate (Capryol PGMC), lecithin, sorbitan monostearate, diethylenglycol monostearate, glyceryl monostearate, and the like, polyoxylglycerides like lauroyl macrogolglycerides (known as GELUCIRE), oleoyl macrogolglycerides (known as LABRAFIL) and caprylocaproyl macrogolglycerides (known as LABRASOL); phospholipid, and mixtures thereof. Preferably, poloxamer and hydrophilic grade of GELUCIRE (e.g., GELUCIRE 44/14) have been found to be useful in the context of present invention.

The complexing agents suitable for use in this invention as hydrophilic carriers include but are not limited to, alpha-cyclodextrin, beta-cyclodextrin, gamma-cyclodextrin, hydroxypropyl beta-cyclodextrin, sulphobutyl ether beta cyclodextrin neutralized poly(acrylic acid), crosslinked acrylic acid copolymers (such as Indion 414), sodium polystyrene sulfonate (such as Amberlite IRP-69), copolymers of methyacrylic acid crosslinked with divinylbenzene (such as Amberlite IRP-64) and polacrilin potassium; zinc acetate, calcium acetate, magnesium acetate, and mixtures thereof.

The cosolvents suitable for use in this invention as hydrophilic carriers include but are not limited to, ethanol, propanol, isopropanol, propylene glycol, polyethylene glycol, dichloromethane, acetone, hexane polyol esters of fatty acids, trialkyl citrate esters, propylene carbonate, dimethylisosorbide, ethyl lactate, N-methylpyrrolidones, transcutol, glycofurol, decaglycerol mono-, dioleate (Caprol PGE-860), triglycerol monooleate (Caprol 3GO), polyglycerol oleate (Caprol MPGO), mixed diesters of Caprylic/Capric acid and propylene glycol (Captex 200), glyceryl mono- and dicaprate (Capmul MCM), isostearyl isostearate, oleic acid, peppermint oil, oleic acid, soybean oil, safflower oil, corn oil, olive oil, cottonseed oil, arachis oil, sunflowerseed oil, palm oil, rapeseed oil, ethyl oleate, glyceryl monooleate, Vitamin E TPGS, alpha tocopherol acetate and mixtures thereof.

The polymer suitable for use in this invention as hydrophilic carriers include but are not limited to, polyvinyl pyrrolidone, hydroxypropyl cellulose, hydroxypropyl methylcellulose, other cellulosic derivatives; hydrocolloids (such as gums), carrageenan and mixtures thereof.

The pharmaceutical composition of the present invention may further include at least one other excipient, non-limiting examples of which include diluents, such as microcrystalline cellulose (“MCC”), silicified MCC (e.g., PROSOLV™), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; cores/beads such as insoluble inert materials like glass particles/beads or silicon dioxide, calcium phosphate dihydrate, dicalcium phosphate, calcium sulfate dihydrate, microcrystalline cellulose, cellulose derivatives; soluble cores such as sugar spheres of sugars like dextrose, lactose, mannitol, starches, sorbitol, or sucrose; insoluble inert plastic materials such as spherical or nearly spherical core beads of polyvinyl chloride, polystyrene or any other pharmaceutically acceptable insoluble synthetic polymeric material, and the like or mixtures thereof; binders or adherents such as acacia, guar gum, alginic acid, dextrin, maltodextrin, methylcellulose, ethyl cellulose, hydroxyethyl cellulose, hydroxypropyl cellulose (e.g., KLUCEL®), low substituted hydroxypropyl cellulose, hydroxypropyl methylcellulose (e.g., METHOCEL®), carboxymethyl cellulose sodium, povidone (various grades of KOLLIDON®, PLASDONE®), starch and the like; disintegrants such as carboxymethyl cellulose calcium, croscarmellose sodium, (e.g., Ac-Di-Sol®, PRIMELLOSE®), crospovidone (e.g., KOLLIDON®, POLYPLASDONE®), povidone K-30, polacrilin potassium, starch, pregelatinized starch, sodium starch glycolate (e.g., PRIMOGEL, EXPLOTAB®), and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, coleyl alcohol, myristyl alcohol and the like. Solvents that may be used in granulation or layering include water, methanol, ethanol, isopropyl alcohol, acetone, methylene chloride, dichloromethane, and the like and mixtures thereof.

Pharmaceutical formulations described herein may further include any one or more of pharmaceutically acceptable glidants and lubricants like stearic acid, magnesium stearate, zinc stearate, talc, colloidal silicon dioxide, sodium stearyl fumarate, opacifiers, colorants, and other commonly used carriers. Suitable preservatives include, by way of example and without limitation, phenoxyethanol, parabens such as methyl paraben and propyl paraben and their sodium salts, propylene glycols, sorbates, urea derivatives such as diazolindinyl urea, and the like and mixtures thereof. Suitable buffering agents include, by way of example and without limitation, sodium hydroxide, potassium hydroxide, ammonium hydroxide and the like and mixtures thereof. Suitable chelating agents include mild agents, such as, for example, ethylenediaminetetraacetic acid (“EDTA”), disodium edetate and EDTA derivatives, and the like and mixtures thereof.

Suitable polymers as excipients include, by way of example and without limitation, those known to one of ordinary skill in the art such as gum arabic, sodium based lignosulfonate, methyl methacrylate, methacrylate copolymers, isobutyl methacrylate, ethylene glycol dimethacrylate, and the like and mixtures thereof.

Suitable gelling agents/viscosifying agents include, by way of example and without limitation, carbomers (carbopol), modified cellulose derivatives, naturally-occurring, synthetic or semi-synthetic gums such as xanthan gum, acacia and tragacanth, sodium alginate, gelatin, modified starches, cellulosic polymers such as hydroxypropyl cellulose, hydroxyethyl cellulose, hydroxypropyl methylcellulose, hydroxypropyl methylcellulose phthalate, and methyl cellulose; co-polymers such as those formed between maleic anhydride and methyl vinyl ether, colloidal silica and methacrylate derivatives, polyethylene oxides, polyoxyethylene-polyoxypropylene copolymers, polyvinyl alcohol and the like and mixtures thereof.

The pharmaceutical composition described herein may further contain one or more suitable solvents. The solvents may appear in the composition or may be used in the preparation of the composition. Examples of such solvents include, but are not limited to, water; tetrahydrofuran; propylene glycol; liquid petrolatum; ether; petroleum ether; alcohols, e.g., methanol, ethanol, isopropyl alcohol and higher alcohols; aromatics, e.g., toluene; alkanes, e.g., pentane, hexane and heptane; ketones, e.g., acetone and methyl ethyl ketone; chlorinated hydrocarbons, e.g., chloroform, carbon tetrachloride, methylene chloride and ethylene dichloride; acetates, e.g., ethyl acetate; lipids, e.g., isopropyl myristate, diisopropyl adipate and mineral oil and the like and mixtures thereof.

In an embodiment, the present invention relates to a pharmaceutical composition for oral administration comprising Compound I or its salt, and a hydrophilic carrier, wherein the pharmaceutical composition releases at least 75% of the contained Compound I or its salt within 60 minutes when tested in USP apparatus type II containing 900 mL of 0.1N HCl with 1% (w/v) SLS maintained at a temperature of about 37±0.5° C., and stirred at 75 rpm. Preferably, the pharmaceutical composition releases at least 85% of the contained Compound I or its salt under the stipulated conditions. The percent (%) active released is measured using HPLC method in comparison with a standard solution.

In another embodiment, the present invention relates to a pharmaceutical composition, wherein the Compound I or its salt is present in partially amorphous form. In an embodiment, the pharmaceutical composition contains at least about 10% of the contained Compound I or its salt in amorphous form. Preferably, the pharmaceutical composition contains from about 10% to about 50%, or more preferably from about 15% to about 40%, of the contained Compound I or its salt in amorphous form.

In an embodiment, the present invention relates to a method of treating a disease condition associated with TRPV3 receptor modulation in a subject wherein the method includes administering the subject a pharmaceutical composition comprising an active ingredient selected from Compound I, Compound II, Compound III, Compound IV and Compound V or salt thereof, and a hydrophilic carrier. The pharmaceutical composition may be in form of a solid dispersion. Preferably, the active ingredient is Compound I or its salt.

In another embodiment, the present invention relates to a method of treating a disease condition associated with TRPV3 receptor modulation in a subject, wherein the method includes administering the subject a pharmaceutical composition comprising Compound I or its salt; and a hydrophilic carrier.

In another embodiment, the present invention contemplates use of a pharmaceutical composition for the treatment of disease condition associated with TRPV3 receptor modulation in a subject comprising administering to the subject a pharmaceutical composition comprising Compound I or its salt and a hydrophilic carrier.

In a further embodiment, the present invention relates to a pharmaceutical composition for the treatment of disease condition associated with TRPV3 receptor modulation in a subject comprising administering to the subject a composition comprising a solid dispersion that includes Compound I or its salt and a hydrophilic carrier.

Non-limiting examples of the disease condition associated with TRPV3 receptor modulation, in the context of present invention, include inflammation, irritable bowel syndrome, Crohn's disease, psoriasis, eczema, dermatitis, post-herpetic neuralgia (shingles), incontinence, bladder incontinence, overactive bladder, bladder cystitis, fever, hot flashes, cough, migraine, arthralgia, cardiac pain arising from an ischemic myocardium, acute pain, chronic pain, neuropathic pain, post-operative pain, pain due to neuralgia (e.g., post-herpetic neuralgia or trigeminal neuralgia), pain due to diabetic neuropathy, dental pain and cancer pain, inflammatory pain conditions (e.g., arthritis and osteoarthritis), myasthenic syndrome, NIDDM and breast cancer.

In yet another embodiment, the present invention provides a process for the preparation of a pharmaceutical composition, said process comprising preparing a solid dispersion of the Compound I or its salt and a hydrophilic carrier; and formulating the solid dispersion in a suitable dosage form. The process comprises preparing a solid dispersion of the Compound I or its salt and a hydrophilic carrier, converting this solid dispersion in a granule formulation and formulating the granules into a suitable dosage form for oral administration.

Preferably, the process comprises dispersing the pyrimidineone derivative in a hydrophilic carrier using techniques such as hot-melt dispersion, spray-drying, granulation and coating.

The granules can be formed by any known processes, using operations such as one or more of dry granulation, wet granulation, and extrusion-spheronization. In an embodiment, the granulation is carried out in equipment such as a planetary mixer, rapid mixer granulator (RMG), fluid bed processor and the like. A fluid bed processor with top or bottom spray attachment has been found to be particularly useful. In general granulation can be carried out by dissolving or dispersing the active ingredient in an organic solvent, optionally with a binder and/or solubilizer, and spraying the solution onto a substrate comprising pharmaceutically acceptable excipients. The granules obtained may further be compressed into tablets or filled in the capsules using techniques known in the art. Alternatively, tablets can be prepared by a direct compression technique using powder blends.

The pharmaceutical compositions of the present invention can be prepared by various other processes and techniques as known to the skilled person so as to achieve desired in vitro drug release profile. Specific embodiments of processes comprise any of:
1. Direct compression, using appropriate punches and dies; the punches and dies being fitted to a suitable rotary tableting press.
2. Injection or compression molding using suitable molds fitted to a compression unit.
3. Granulation followed by compression.
4. Extrusion in the form of a paste, into a mold or to an extrudate to be cut into lengths.
5. Formation of solid dispersion by solvent evaporation.
6. Formation of solid dispersion by hot melt technique.
7. Preparation of suspension by high pressure homogenization.

The following examples are provided to enable one skilled in the art to practice the invention and are merely illustrative of the invention. The examples should not be read as limiting the scope of the invention.

### EXAMPLES

### Example 1

### Solubility Data of Compound I with Various Hydrophilic Carriers and in Solid Dispersion Composition Form at 25° C.

Compound I and hydrophilic carriers were mixed as per ratio provided in Table 1. Buffers were prepared as per USP. Compound I (100 mg) equivalent was added to various buffers (100 ml) in presence of various hydrophilic carriers. The samples were sonicated for 15 minutes. A solid dispersion composition form of Compound I was also evaluated for solubility on similar lines. Quantification was done by assay method on HPLC by comparison with standard solution. The assay provided the relative amount of drug (mg/ml) in the sample solution. The results were extrapolated to represent the solubility in 900 ml of buffer solutions. The data thus generated is provided in Table 1.

TABLE 1
Solubility of Compound I

| | Compound I | Solubility (mg/900 ml) | | |
|---|---|---|---|---|
| | to hydrophilic | Simulated | Acetate | Phosphate |
| Hydrophilic carrier/ | carrier | gastric fluid | Buffer | Buffer |
| Composition | ratio (w/w) | pH 1.2 | pH 4.5 | pH 6.8 |
| None (without | 1:0 | BLD** | BLD | BLD |
| hydrophilic carrier) | | | | |
| Poloxamer 407 | 1:1 | 0.27 | 0.18 | 0.09 |
| Labrasol* | 1:10 | 0.36 | 0.18 | 0.72 |
| Labrafil* | 1:10 | 0.09 | 0.27 | 0.18 |
| Gelucire* 44/14 | 1:10 | 0.09 | 1.17 | 0.99 |
| Vitamin E TPGS | 1:10 | 0.63 | 1.35 | 1.80 |
| Cetrimide | 1:10 | 0.54 | 0.27 | 0.45 |
| Tween 80 | 1:10 | 0.45 | 3.06 | 3.69 |
| Tween 80 | 1:30 | 1.44 | 8.82 | 15.84 |
| HPMC | 1:5 | 0.72 | 0.18 | 0.81 |
| Cremophor EL | 1:10 | 0.54 | 2.61 | 2.52 |
| Sodium lauryl | 1:5 | BLD | BLD | BLD |
| sulphate | | | | |
| Hydroxypropyl | 1:10 | BLD | BLD | BLD |
| beta-cyclodextrin | | | | |
| Solid dispersion | | 4.86 | 4.59 | 6.48 |
| of Compound I*** | | | | |

*Commercially available from Gattefosse Ltd.
**Below limit of detection (Limit of detection: 0.01 ppm)
***Granules of Example 7 prior to filling into capsules.

### Brief Analytical Method for Determining Solubility of Compound I:

Preparation of Test solution: Compound I (100 mg) was added to various buffers (100 ml) in presence of various surfactants. The samples were sonicated for 15 minutes. Evaluation was done by assay method on HPLC by comparison with standard solution.

### Mobile Phase:

Mixture of 0.01M Ammonium Acetate Buffer and Acetonitrile in the ratio of 20:80% v/v.

### Diluent:

Mixture of water and acetonitrile in the ratio of 20:80 v/v.

### Chromatographic Conditions:

Column: Hypersil BDS C18, 150×4.6 mm, 5 μm

Flow Rate: 1.0 ml/minute

Detection: UV at 330 nm

Column temperature: 25° C.

Injection volume: 50 μl

Run time: 10 minutes

Retention Time: About 5 minutes

### Example 2

### Pharmaceutical Composition Containing Compound I and Various Hydrophilic Carriers

| Ingredients | Composition (% w/w) |
|---|---|
| Compound I | 7.14 |
| Poloxamer 407 | 14.29 |
| Hydroxypropyl methylcellulose | 7.14 |
| Sugar spheres (40#-60#) | 64.29 |
| Gelucire 44/14 | 7.14 |
| Isopropyl alcohol | q.s. |
| Dichloromethane | q.s. |

### Manufacturing Process:

1. Hydroxypropyl methylcellulose was dispersed in Isopropyl alcohol under stirring.
2. Dichloromethane was added to the dispersion of Step 1 under stirring.
3. Compound I was dissolved in a mixture of dichloromethane and isopropyl alcohol.
4. Poloxamer 407 was added to the solution of Step 3 under stirring to obtain a clear dispersion.
5. Gelucire 44/14 was heated to melt on a water-bath and was added to the dispersion of Step 4.
6. The dispersion of Step 5 was added to the dispersion of Step 2 under stirring to obtain a clear dispersion.
7. The dispersion of Step 6 was sprayed onto sugar spheres in a fluid bed processor to obtain granules.
   In vitro dissolution data using USP dissolution test apparatus (Type II) in 900 ml of 0.1 N HCl, stirred at 50 rpm.

| Time (min) | Cumulative drug dissolved (%) |
|---|---|
| 0 | 0 |
| 15 | 7.3 |
| 30 | 8.0 |

### Brief Analytical Process for the Dissolution of Compound I:

The amount of Compound I dissolved was determined by HPLC method by comparison with standard solution,

### Mobile Phase:

Mixture of 0.01M Ammonium Acetate Buffer and Acetonitrile in the ratio of 20:80% v/v.

### Diluent:

Mixture of water and acetonitrile in the ratio of 20:80 v/v.

### Chromatographic Conditions:

Column: Hypersil BDS C18, 150×4.6 mm, 5 μm

Flow Rate: 1.0 mL/minute.

Detection: UV at 330 nm.

Column temperature: 25° C.

Injection volume: 50 μl.

Run time: 10 minutes.

Retention Time About 5 minutes.

### Stability Data:

The granules were stored in triple laminated pouches, each pouch containing about 50 gm of granules. Each pouch was packed in a HPDE container and stored under different storage conditions. The percent dissolution at 60 min at various storage intervals was evaluated.

In vitro dissolution data after 60 min using USP dissolution test apparatus (Type II) in 900 ml of 0.1 N HCl with 1% sodium lauryl sulphate stirred at 75 rpm.

| | Cumulative drug dissolved (%) | | | | |
|---|---|---|---|---|---|
| Storage condition | Initial | 1 Month | 2 Months | 3 Months | 6 Months |
| 2 to 8° C. | 96 | — | — | 97 | 99 |
| 25 ± 2° C. and | 96 | — | — | 101 | 101 |
| 60 ± 5% RH | | | | | |
| 30 ± 2° C. and | 96 | — | — | 99 | 101 |
| 65 ± 5% RH | | | | | |
| 40 ± 2° C. and | 96 | 99 | 102 | 96 | 102 |
| 75 ± 5% RH | | | | | |

### Examples 3-5

### Pharmaceutical Capsule Compositions Containing Compound and Various Hydrophilic Carriers

| | Quantity per capsule (mg) | | |
|---|---|---|---|
| Ingredients | Example 3 | Example 4 | Example 5 |
| Compound I | 30 | 30 | 30 |
| Labrasol | — | — | 70 |
| Gelucire 44/14 | — | — | 218 |
| Vitamin E TPGS | 300 | — | 300 |
| PEG 4000 | 200 | 300 | 400 |
| Poloxamer 407 | 100 | 100 | — |
| Gelucire 50/13 | — | 200 | — |

Manufacturing process for Example 3:
1. Vitamin E TPGS, PEG 4000 and Poloxamer 407 were mixed together at 45° C. and stirred continuously to obtain a dispersion.
2. Compound I was added to the dispersion of Step 1 maintained at about 45° C. and stirred continuously to obtain a uniform dispersion.
3. The dispersion of Step 2 was maintained in its molten state and was filled into capsules.

### Manufacturing Process for Example 4:

1. PEG 4000, Poloxamer 407 and Gelucire 50/13 were mixed together at 45° C. and stirred continuously to obtain dispersion.
2. Compound I was added to the dispersion of Step 1 maintained at about 45° C. and stirred continuously to obtain a uniform dispersion.
3. The dispersion of Step 2 was maintained in its molten state and was filled into capsules.

### Manufacturing Process for Example 5:

1. Labrasol was heated to about 45° C. and was maintained at the same temperature.
2. Gelucire 44/14, Vitamin E TPGS and PEG 4000, were added to the molten material of Step 1 and stirred continuously maintaining the temperature to obtain a molten dispersion.
3. Compound I was added to the dispersion of Step 2 maintained at about 45° C. and stirred continuously to obtain a uniform dispersion.
4. The dispersion of Step 3 was maintained in its molten state and was filled into capsules.

In vitro dissolution data using USP dissolution test apparatus (Type II) in 900 ml of 0.1 N HCl, stirred at 50 rpm.

| | Cumulative drug dissolved (%) | | |
|---|---|---|---|
| Time (min) | Example 3 | Example 4 | Example 5 |
| 0 | 0 | 0 | 0 |
| 15 | 24.8 | 24.5 | 58.1 |
| 30 | 26.2 | 42.4 | 89.1 |

### Example 6

### Pharmaceutical Composition in the Form of Granules Filled in a Capsule Containing Compound I and Various Hydrophilic Carriers

| Ingredients | Quantity per capsule (mg) |
|---|---|
| Compound I | 30 |
| Labrasol | 70 |
| Gelucire 44/14 | 218 |
| Vitamin E TPGS | 300 |
| PEG 4000 | 400 |
| Sugar spheres (40#-60#) | 500 |

### Manufacturing Process:

1. Labrasol was heated to melt at about 45° C. and was maintained at the same temperature.
2. Gelucire 44/14, Vitamin E TPGS and PEG 4000 were dispersed in the melt of Step 1 and stirred continuously while maintaining the temperature at about 45° C.
3. Compound I was added to the dispersion of Step 2 and stirred continuously while maintaining the temperature at about 45° C. to obtain a uniform dispersion.
4. The dispersion of Step 3 was adsorbed onto the sugar spheres to obtain a semisolid mass.
5. The mass of Step 4 was passed through ASTM Sieve #16 to obtain granules.
6. The granules of Step 5 were dried and compressed to form tablet.
7. Alternately, the granules of Step 5 were dried and filled into hard gelatin capsule.
   In vitro dissolution data using USP dissolution test apparatus (Type II) in 900 ml of 0.1 N HCl, stirred at 50 rpm.

| Time (min) | Cumulative drug dissolved (%) |
|---|---|
| 0 | 0 |
| 15 | 24.1 |
| 30 | 30.4 |

### Example 7

### Pharmaceutical Composition in the Form of Granules Filled in a Capsule Containing Compound I and Various Hydrophilic Carriers

| Ingredients | Quantity per capsule (mg) |
|---|---|
| Compound I | 30 |
| Poloxamer 407 | 60 |
| HPMC E5LV | 30 |
| NP seeds (40#-60#) | 270 |
| Gelucire 44/14 | 30 |
| Isopropyl alcohol | q.s. |
| Methylene chloride | q.s. |

### Manufacturing Process:

1. HPMC E5LV was dispersed under stirring in sufficient quantity of isopropyl alcohol.
2. A part of the methylene chloride was added to the dispersion of Step 1 under slow stirring.
3. Compound I was dispersed in the remaining part of the methylene chloride.
4. Gelucire 44/14 was melted and added to Poloxamer 407 and mixed.
5. The dispersion of Step 3 was dispersed in the dispersion of step 4 under stirring.
6. The dispersion of Step 5 was sprayed onto the NP seeds in a fluid bed processor and the granules thus obtained were filled in capsules.

In vitro dissolution data using USP dissolution test apparatus (Type II) in 900 ml of 0.1 N HCl stirred at 50 rpm.

| | Cumulative drug | Cumulative Drug |
|---|---|---|
| Time (min) | dissolved (%)* | dissolved (%)** |
| 0 | 0 | 0 |
| 15 | 7.2 | 52.5 |
| 30 | 7.4 | 54.9 |

*The sample was filtered through 0.45 μm pore size filter.
**The sample was filtered through 10 μm pore size filter.

X-Ray Diffraction studies were carried out on Compound I, placebo granule composition of Example 7, and granule composition of Example 7. The studies were carried out on a PANalytical X-ray diffractometer (Model: X′Pert Pro).

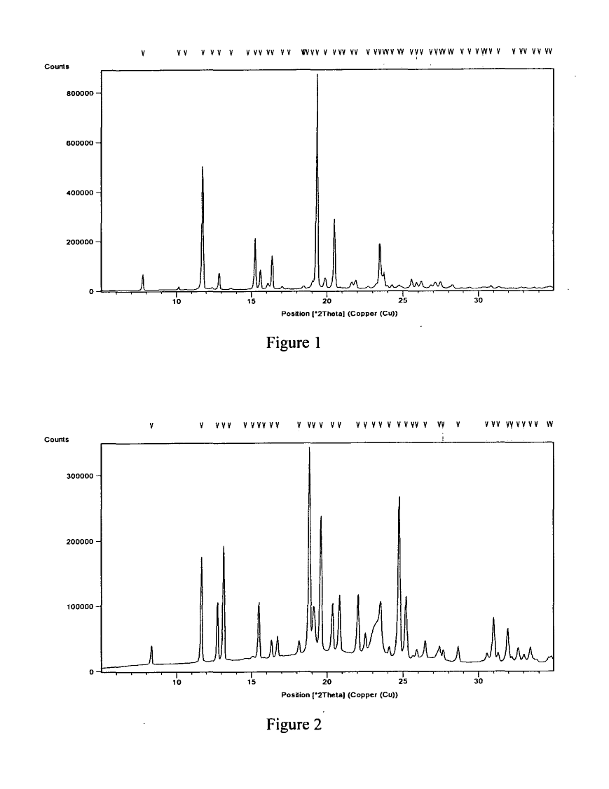

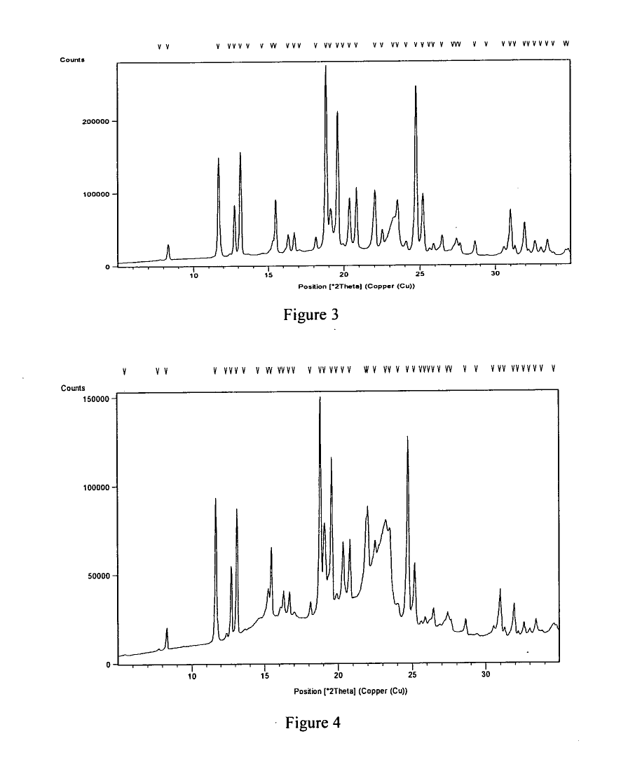

The X-ray Diffraction studies of Compound I is depicted in FIG. 1. The XRD of placebo granule composition of Example 7 is represented in FIG. 2 and that of the granule composition Example 7 is given in FIG. 3.

Thus, it seems that the granules composition of Example 7 comprises from about 15% to about 20% of the contained Compound I or its salt in amorphous form.

### Example 8

### Pharmaceutical Composition in the Form of Oral Solution Containing Compound I and Various Hydrophilic Carriers

| Ingredients | Composition (% w/v) |
|---|---|
| Compound I | 0.90 |
| Labrasol | 5.00 |
| PEG 400 | 6.00 |
| Cremophor EL | 23.00 |
| Tween 80 | 4.00 |
| Propylene Glycol | 55.20 |
| Water | 5.50 |
| Sodium Saccharin | 0.40 |
| Propylene Glycol | q.s |
| Strawberry flavor | q.s |

### Manufacturing Process:

1. Labrasol, PEG 400 and Cremophor EL were mixed and continuously stirred to obtain a uniform dispersion.
2. Compound I was added to the dispersion of Step 1 and was continuously stirred maintaining temperature at about 45° C. to obtain a solution.
3. Tween 80, a part of propylene glycol and sodium saccharin were added to the solution of Step 2 and stirred continuously maintaining temperature of about 45° C. to obtain a uniform dispersion.
4. Water was added slowly under continuous stirring to the dispersion of Step 3, maintaining the temperature at about 45° C. to obtain a solution.
5. The solution of Step 4 was cooled to room temperature.
6. Strawberry flavor was added and volume was made up with propylene glycol.

In vitro dissolution data of oral solution equivalent to 30 mg of Compound I using USP dissolution test apparatus (Type II) in 900 ml of 0.1 N HCl stirred at 50 rpm.

| Time (min) | Cumulative drug dissolved (%) |
|---|---|
| 0 | 0 |
| 15 | 81.88 |
| 30 | 85.1 |

### Example 9

### Pharmaceutical Composition Containing Compound I and Various Hydrophilic Carriers

| Ingredients | Composition (% w/v) |
|---|---|
| Compound I | 0.2 |
| Gelucire 44/14 | 32 |
| Labrasol | 47 |
| Vitamin E TPGS | 2 |
| Propylene glycol | q.s. |

### Manufacturing Process:

1. Labrasol was heated to melt at about 45° C. and was maintained at the same temperature.
2. Gelucire 44/14 and Vitamin E TPGS were dispersed in the melt of Step 1 and stirred continuously while maintaining the temperature at about 45° C.
3. Compound I was added to the dispersion of Step 2 and stirred continuously while maintaining the temperature at about 45° C. to obtain a uniform dispersion.
4. The dispersion of Step 3 was maintained in its molten state and was filled into capsules.

### Example 10

### Pharmaceutical Composition Containing Compound I and Various Hydrophilic Carriers

| Ingredients | Composition (% w/v) |
|---|---|
| Compound I | 0.2 |
| PEG 4000 | 10 |
| Propylene glycol | 10 |
| Glycerin | 20 |
| Ethanol | 1 |
| 0.5% methyl cellulose suspension | q.s. |

### Manufacturing Process:

1. Propylene glycol, glycerin and ethanol were mixed together under continuous stirring at about 45° C. to obtain a uniform dispersion.
2. PEG 4000 was added to the dispersion of Step 1 under continuous stirring at 45° C. to obtain a uniform dispersion.
3. Compound I was added and mixed to the dispersion of Step 2 maintained at a temperature of about 45° C. to obtain a uniform dispersion.
4. 0.5% methyl cellulose suspension was added to the dispersion of Step 3 slowly under continuous stirring till a uniform dispersion was obtained and the dispersion was cooled to room temperature.

### Comparative Examples A-D

### Tablet or Capsule Containing the Granules of Compound I

| | mg per tablet or capsule | | | |
|---|---|---|---|---|
| | Comparative | Comparative | Comparative | Comparative |
| | Example A | Example B | Example C | Example D |
| Ingredients | | | | |
| Compound I | 10.0 | 10.0 | 10.0 | 10.0 |
| Sodium lauryl | 25.0 | — | — | — |
| sulphate | | | | |
| Pearlitol SD | 165.0 | 190.0 | 179.0 | — |
| 200 | | | | |
| Docusate | — | — | 11.0 | — |
| sodium | | | | |
| Eudragit EPO | — | — | — | 10.0 |
| Isopropyl | q.s | — | q.s | — |
| alcohol | | | | |
| Dichloro- | q.s | — | q.s | — |
| methane | | | | |
| Water purified | — | q.s. | — | — |
| Total (mg) | 200 | 200 | 200 | 20 |
| In vitro dis- | | | | |
| solution data* | | | | |
| 0 min | 0 | 0 | 0 | 0 |
| 15 min | 1.8 | 1.6 | 4.3 | 1.6 |
| 30 min | 1.6 | 1.8 | 5.0 | 1.5 |

*For granules equivalent to 10 mg of Compound I using USP dissolution test apparatus (Type II) in 900 ml of 0.1N HCl stirred at 50 rpm.

### Example 11

### Pharmaceutical Composition Containing Compound I Composition in the Form of Tablets

| | |
|---|---|
| Ingredients | Quantity per tablet (mg) |

Granules composition

| | |
|---|---|
| Compound I | 30 |
| Poloxamer 407 | 30 |
| Hypromellose 5 cps | 30 |
| Gelucire 44/14 | 60 |
| Sugar spheres (#120-140) | 156 |
| Isopropyl alcohol | q.s. |
| Dichloromethane | q.s. |
| Total (Granule composition) | 306 |

Tablet composition

| | |
|---|---|
| Granules composition | 306 |
| Avicel 102* | 138.5 |
| Ac-di-sol | 50 |
| Aerosil | 2.75 |
| Mag. stearate | 2.75 |

*Adjusted based on Assay of granules

### Manufacturing Process:

1. Hypromellose was dispersed in isopropyl alcohol with constant stirring.
2. Dichloromethane was added to the dispersion of step 1 and stirred till clear solution was obtained.
3. Compound I was dissolved in above solution with constant stirring.
4. Gelucire 44/14 and Poloxamer 407 were melted and added with constant stirring to the above solution.
5. The mixture of step 4 was sprayed onto sugar spheres in a fluid bed processor using bottom spray.
6. Dried granules were sifted through #30 ASTM and blended with Avicel 102, Ac-di-sol and Aerosil and finally lubricated with Magnesium stearate.
7. The above granules of step 6 were formulated into tablets and the thus formed tablets were suitably coated.
   In vitro dissolution data using USP dissolution test apparatus (Type II) in 900 ml of 0.1 N HCl, stirred at 50 rpm.

| Time (min) | % drug released |
|---|---|
| 15 | 7.6 |
| 30 | 10.1 |
| 60 | 11.5 |

### Stability Study:

The tablets were subjected to accelerated stability conditions. The amount of Compound I dissolved was determined by HPLC method in comparison with standard solution.

### Chromatographic Conditions:

### Column: Hypersil BDS C-18, 150×4.6 mm, 5 μm

Flow Rate: 1.0 ml/minute

### Detection: UV 330 nm

Column temperature: 25° C.
Injection volume: 50 μl
Run time: 10 minutes
Retention Time: About 5 minutes
The in vitro dissolution studies were carried out using USP II (Paddle apparatus) in 900 ml of dissolution media, i.e. 0.1N HCl with 1% (w/v) SLS at 37±0.5° C.; at 75 rpm for 60 minutes.

| Storage condition | Initial | 25° C. 60% RH | 40° C. 75% RH |
|---|---|---|---|
| Test Parameter | Initial | 3 Months | 3 Months |
| Dissolution in 0.1N HCl | 98.2 | 97.4 | 98.3 |
| (% drug released) | | | |

The related substances (i.e. single maximum impurity and total impurity) was determined using HPLC and the Assay was performed (by HPLC) under storage conditions.

| Storage condition | Initial | 25° C. 60% RH | 40° C. 75% RH |
|---|---|---|---|
| Test Parameter | Initial | 3 Months | 3 Months |
| Single maximum | 0.08 | 0.06 | 0.07 |
| impurity (% w/w) | | | |
| Total impurities (% w/w) | 0.25 | 0.25 | 0.25 |
| Assay by HPLC (% w/w) | 103.3 | 102.0 | 102.1 |

X-Ray Diffraction studies were carried out on the pharmaceutical composition of Example 11. The studies were carried out on a PANalytical X-ray diffractometer (Model: X′Pert Pro). The X-Ray diffraction pattern is represented in FIG. 4.

### Example 12

### Systemic Exposure Studies of Compositions of Compound I in Dogs

Systemic exposure studies were carried out in beagle dogs at a dose of 10 mg/kg as a single dose administration. The animals were dosed by oral gavage. The pharmacokinetic data obtained is provided in Table 2.

TABLE 2
Pharmacokinetic data of various examples.

| | Compositions | | | |
|---|---|---|---|---|
| Pharmacokinetic | Example 4 | Example 6 | Example 8 | Example 9 |
| parameter | (n = 3) | (n = 3) | (n = 4) | (n = 4) |
| Cₘₐₓ (ng/ml) | 832.94 | 502.68 | 928.75 | 312.52 |
| AUC₍₀₋₂₄₎ (ng · hr/ml) | 7616.15 | 4357.51 | 7984.59 | 951.76 |
| AUC_{(0-inf)} (ng · hr/ml) | 8384.79 | 4588.80 | 12346.10 | 1072.77 |
| Tₘₐₓ (hour) | 3.67 | 2.50 | 6.00 | 1.125 |

All publications, patents, and patent applications cited in this application are herein incorporated by reference to the same extent as if each individual publication, patent, or patent application was specifically and individually indicated to be incorporated herein by reference.

1. A pharmaceutical composition comprising a solid dispersion that includes an active ingredient 7-{(E)-2-[2-(cyclopropylmethoxy)-3-methoxyphenyl]vinyl}-6-(4-trifluoromethoxy-phenyl)-5H-[1,3]thiazolo-[3,2-a]pyrimidin-5-one (“Compound I”) or its salt; and a hydrophilic carrier.

2. (canceled)

3. A pharmaceutical composition according to claim 1, wherein the hydrophilic carrier includes surfactant, complexing agent, cosolvent, polymer and mixtures thereof.

4. A pharmaceutical composition according to claim 1, wherein the hydrophilic carrier is a surfactant selected from poloxamer, polyoxyethylene sorbitan ester, polyoxyethylene sorbitan ester, polyglyceryl-6-dioleate, polyethylene glycol 15 hydroxy stearate, diethylene glycol monoethyl ether, propylene glycol dicaprylocaprate, lecithin, phospholipid, lauroyl macrogolglycerides, oleoyl macrogolglycerides, and caprylocaproyl macrogolglycerides, or mixtures thereof.

5. A pharmaceutical composition according to claim 1, wherein the hydrophilic carrier is a complexing agent selected from alpha-cyclodextrin, beta-cyclodextrin, gamma-cyclodextrin, hydroxypropyl beta-cyclodextrin, sulphobutyl ether beta cyclodextrin neutralized poly(acrylic acid), and crosslinked acrylic acid copolymers, or mixtures thereof.

6. A pharmaceutical composition according to claim 1, wherein the hydrophilic carrier is a cosolvent selected from ethanol, propanol, isopropanol, propylene glycol, polyethylene glycol, dichloromethane, dimethylisosorbide, ethyl lactate, N-methylpyrrolidones, glycofurol, decaglycerol mono-, dioleate, triglycerol monooleate, polyglycerol oleate, mixed diesters of Caprylic/Capric acid and propylene glycol, ethyl oleate, glyceryl monooleate, Vitamin E TPGS, alpha tocopherol, or mixtures thereof.

7. A pharmaceutical composition according to claim 1, wherein the hydrophilic carrier is a polymer selected from polyvinyl pyrrolidone, hydroxypropyl cellulose, hydroxypropylmethyl cellulose, hydroxyethyl cellulose, carrageenan, or mixtures thereof.

8. (canceled)

9. A pharmaceutical composition according to claim 1, wherein the weight ratio of the Compound I or its salt to the hydrophilic carrier ranges from about 1:0.5 to about 1:50.

10. A pharmaceutical composition according to claim 1, wherein the weight ratio of the Compound I or its salt to the hydrophilic carrier ranges from about 1:1 to about 1:20.

11. A pharmaceutical composition according to claim 1, wherein the weight ratio of the Compound I or its salt to the hydrophilic carrier ranges from about 1:1 to about 1:10.

12. (canceled)

13. A pharmaceutical composition according to claim 1, wherein the hydrophilic carrier is selected from poloxamer, lauroyl macrogolglycerides and hydroxypropylmethyl cellulose, or mixtures thereof, and wherein the weight ratio of the Compound I or its salt to the hydrophilic carrier ranges from about 1:1 to about 1:10.

14. (canceled)

15. (canceled)

16. (canceled)

17. A pharmaceutical composition according to claim 1, wherein Compound I or its salt is present in an amount ranging from about 1% w/w to about 50% w/w.

18. (canceled)

19. (canceled)

20. (canceled)

21. (canceled)

22. (canceled)

23. (canceled)

24. (canceled)

25. A method of treating a disease condition associated with TRPV3 receptor modulation in a subject, said method comprising administering to the subject a pharmaceutical composition comprising a solid dispersion that includes Compound I or its salt; and a hydrophilic carrier, wherein the hydrophilic carrier includes surfactant, complexing agent, cosolvent, polymer and mixtures thereof.

26. (canceled)

27. (canceled)

28. A process for preparing a pharmaceutical composition comprising a solid dispersion that includes Compound I or its salt; and a hydrophilic carrier, said process comprising: (a) preparing a solid dispersion of the Compound I or its salt and a hydrophilic carrier; and (b) formulating the solid dispersion in a suitable dosage form.

29. A process according to claim 28, wherein the pharmaceutical composition is suitable for oral administration.

30. A method according to claim 25, wherein the weight ratio of the Compound I or its salt to the hydrophilic carrier ranges from about 1:1 to about 1:10.
